(19) United States
(12) Patent Application Publication
(10) Pub. No.: US 2022/0088072 A1
(43) Pub. Date: Mar. 24, 2022

(54) TREATMENT METHOD FOR PSORIASIS

(71) Applicant: REGENEX LLC, Moscow (RU)

(72) Inventor: Svetlana N. BYKOVSKAIA, Woodland Hills, CA (US)

(73) Assignee: REGENEX LLC, Moscow (RU)

(21) Appl. No.: 17/314,987

(22) PCT Filed: Nov. 11, 2019
(86) PCT No.: PCT/IB2019/059674

§ 371 Date: May 7, 2021

Related U.S. Application Data
(60) Provisional application No. 62/758,360, filed on Nov. 9, 2018.

(51) Int. Cl. A61K 35/17; C07K 14/73; C07K 14/725

(52) U.S. Cl. CPC A61K 35/17; C07K 14/7051; C07K 14/70514

(57) ABSTRACT

Disclosed is a method for treating or preventing psoriasis in a patient by administering to the patient autologous, ex vivo-modified and expandedCD4⁺CD25⁺Foxp3⁺CD127^{low} T reg cells.

### PRIORITY CLAIM

This application claims priority to U.S. Provisional application No. 62/758,360, filed Nov. 9, 2018, the contents of which are hereby incorporated herein in their entirety.

### FIELD OF INVENTION

The invention relates to medicine, immunology, and more specifically to the prevention and/or treatment of psoriasis.

### BACKGROUND

Psoriasis is an autoimmune disease that affects two to four percent of the human population and affects men and women equally.

Psoriasis is characterized by an abnormally excessive and rapid growth of the epidermal layer of the skin. Abnormal production of skin cells (especially during wound repair) and an overabundance of skin cells result from the sequence of pathological events in psoriasis. It is characterized by patches of abnormal skin which are typically red, dry, itchy, and scaly, and which may be discolored in people with darker skin color. These regions of abnormalities vary from small, localized patches to complete body coverage.

Psoriasis is believed to be a genetic disease that is triggered by environmental factors. For example, symptoms often worsen during winter and with certain medications, such as beta blockers or NSAIDs. Infections and psychological stress may also be triggers. These periods of increased disease are called flare-ups. There are five main types of psoriasis: plaque, guttate, inverse, pustular, and erythrodermic. Plaque psoriasis, also known as psoriasis vulgaris, is the most common, making up about 90 percent of cases. It typically presents as red patches with white scales on top. Areas of the body most commonly affected are the back of the for psoriasis, shins, navel area, and scalp. Guttate psoriasis has drop-shaped lesions. Pustular psoriasis presents as small non-infectious pus-filled blisters.^{[} Inverse psoriasis for psoriasis red patches in skin folds. Erythrodermic psoriasis occurs when the rash becomes very widespread, and can develop from any of the other types. In most affected people, changes in color of fingernails and toenails often occur.

The underlying mechanism involves the immune system reacting to skin cells. Skin cells are replaced every three to five days in psoriasis rather than the usual 28 to 30 days. These changes are believed to stem from the premature maturation of keratinocytes induced by an inflammatory cascade in the dermis involving dendritic cells, macrophages, and T cells. These immune cells move from the dermis to the epidermis and secrete inflammatory chemical signals (cytokines) such as interleukins, tumor necrosis factor-α, interleukin-1β, interleukin-6, and interleukin-22. These secreted inflammatory signals are believed to stimulate keratinocytes to proliferate.

Presently, there is no cure for psoriasis. However, treatments with topical agents such as steroid creapsoriasis and vitamin D3 cream are used to treat the symptoms of mild disease. Moderate disease is often treated with ultraviolet light, but unfortunately, UV light therapies provide the risk of developing skin cancer.

Systemically administered immune system suppressing medications, such as methotrexate, ciclosporin, hydroxycarbamide, fumarates such as dimethyl fumarate, and retinoids can help to control the symptopsoriasis of severe disease. However, patients undergoing systemic treatment must have regular blood and liver function tests to check for medication toxicities, and pregnancy must be avoided. Systemically applied monoclonal antibodies that target cytokines, such as TNF-α, have also been used. However, individuals with psoriasis may develop neutralizing antibodies against monoclonal antibodies.

Thus, what is needed are better methods of treatment of, and a cure for, psoriasis.

### SUMMARY

It has been discovered that there is an inverse relationship between the number of CD4⁺CD25⁺Foxp3⁺CD27^{low} regulatory T (T reg) cells in the peripheral blood of patients with psoriasis and the degree of disability and severity of the disease. This discovery has been exploited to develop the present method of treating of psoriasis. Administering autologous, ex vivo-modified and expanded T reg cells during the remission phase lowers the level of autoimmune activity of certain T cells, thereby reducing symptoms and maintaining the remission phase indefinitely or for at least longer periods of time than what is seen on average without treatment.

In one aspect, the disclosure provides a method of treaty psoriasis a patient in need thereof, comprising: administering a therapeutically effective amount of autologous, modified and expanded T reg cells to the patient during a remission phase.

In some embodiments, the administering step comprises administering the therapeutically effective amount of these T reg cells more than one time at the start of treatment to increase the number of T reg cells in patient's blood to a number comparable to number in the blood of healthy donors. In some embodiments, the administering step comprises administering a therapeutically effect amount of these T reg cells about 1 to 7 times at the start of treatment.

In some embodiments the method further comprising measuring the number of T reg cells in the blood of the patient before and after each administering step. In particular embodiments, the number of T reg cells in a patient's blood is measured monthly, weekly, or every week, 1 to 3 months, or every 2 to 3 months, after the initial administering step.

In particular embodiments, the method further comprises a second administering step if the number of T reg cells measured in the peripheral blood of a patient after the first administering step is less than the number of T reg levels in peripheral blood of a healthy donor.

In certain embodiments, the number of autologous, modified and expanded T reg cells administered at one time is about 1×10⁶ to about 1.1×10⁷ per kg body weight to the patient. In many embodiments, these T reg cells are administered by subcutaneous, intravenous, and/or intramuscular injection.

Also provided by the present disclosure is a method of inhibiting the activity of autoimmune, autologous, cytotoxic T and B cells in a patient suffering from psoriasis, comprising: administering a therapeutically effective amount of autologous, modified and expanded T reg cells to the patient. In some embodiments, the administering step is performed more than one time throughout the life of the patient. In certain embodiments, the administering step is performed every week, every 1 to 7, 2 to 6, 3 to 6, or 4 to 5 months after the first administering step.

In many embodiments, the administering step comprises administering about 1×10⁶ to about 1.1×10⁷ autologous, modified and expanded T reg cells per kg body weight to the patient. In many embodiments, these T reg cells are administered by subcutaneous, intravenous, and/or intramuscular injection.

### DESCRIPTION OF THE FIGURES

The foregoing and other objects of the present disclosure, the various features thereof, as well as the invention itself may be more fully understood from the following description, when read together with the accompanying drawings in which:

FIG. 1A is a scatter plot showing characteristic T reg markers in the population of donor mononuclear cells which are CD25⁺;

FIG. 1B is a scatter plot showing characteristic T reg markers in the population of donor mononuclear cells which are Foxp3;

FIG. 1C is a scatter plot showing characteristic T reg markers in the population of donor mononuclear cells which are CD27^{low};

FIG. 2A is a scatter plot showing characteristic T reg markers in the population of donor CD4⁺ T cells which are CD25⁺;

FIG. 2B is a scatter plot showing characteristic T reg markers in the population of donor CD4⁺ T cells which are Foxp3;

FIG. 2C is a scatter plot showing characteristic T reg markers in the population of donor CD4⁺ T cells which are CD27^{low};

FIG. 3A is a scatter plot showing the expression of CD25^{hi} on T cells after 6 days of cultivation;

FIG. 3B is a scatter plot showing the expression of Foxp3⁺ on T cells after 6 days of cultivation;

FIG. 3C is a scatter plot showing the expression of CD27^{low} on T cells after 6 days of cultivation; and

FIG. 4 is a graphic representation showing the increase in total number of cells (gray columns) and the increase in the number of T reg cells (shaded columns) at different stages of cultivation.

### DETAILED DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

It has been discovered that the number of T reg cells in a patient suffering from psoriasis is variable, and that there is an inverse relationship between the number of T reg cells in the peripheral blood of such patients and the degree of disability, severity, and duration of the condition. Thus, a reduced level and functional activity of T reg cells are now understood to play an important role in the progression of the disease. On this basis, a promising method for treatment of psoriasis was developed which includes the immune correction therapy comprising T reg cells. In addition, it has been determined that treatment with autologous, modified and expanded T reg cells can inhibit the activity of autoimmune, autologous, cytotoxic T and B cells in a patient suffering from psoriasis.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the term “T reg cells” refers to regulatory T cells with markers CD4⁺CD25⁺Foxp3⁺CD27^{low}.

As used herein, the term “treating” refers to reducing or alleviating the symptom of psoriasis, and/or preventing flare-ups and/or the progression of the disease.

The term “preventing” refers to inhibiting or stopping an affected person with a flare-up or psoriasis.

The term “native” refers to cells from the body that have not been cultured, modified, expanded, or treated with any compound other than a life-sustaining medium.

“Ex vivo-modified and expanded” refers to native cells removed from the body, treated with various factors and compounds to modify their phenotype, and cultivated to proliferate. In the methods of the present disclosure, when cells treated in this way are returned to the body of the patient from which they were originally removed, they are referred to as “autologous, ex vivo-modified and expanded cells”.

As used herein, the term “healthy donor” refers to a mammal, such a human, of the same specie as the patient and who does not have any of psoriasis, does not have any blood-related or inflammatory disorder, and is considered by a physician to be in good health. The number of T reg cells in the blood of a healthy donor is used herein to determine the number of T reg cells that are administered to the patient.

### 2. Preparation of Autologous, Ex Vivo-Modified and Expanded T Reg Cells

Autologous, modified and expanded T reg cells administered to a patient with psoriasis according to the method of the disclosure are derived from the peripheral blood of the patient. A sample of blood is removed and the fractionated to obtain a mononuclear cell (MNC) fraction from which the CD4⁺ T cells are later isolated. The MNC fraction can be obtained from blood by any known method of blood fractionation. For example, density gradient centrifugation, e.g. Ficoll-Hypaque density gradient centrifugation, can be used which takes advantage of the density differences between MNC's and other elements found in the blood sample. MNC's and platelets collect on top of the Ficoll-Hypaque layer because they have a lower density than red blood cells and granulocytes which collect at the bottom of the Ficoll-Hypaque layer.

To obtain T cells with a regulatory function, cells in this mononuclear fraction can be exposed to antibodies specific for CD4, as such T cells test positive for this marker. CD4⁺ cells can then be separated from the MNC fraction, for example, using CD4⁺ MicroBead columns (Myltenyi Biotec, Germany) exposed to a magnetic field. These CD4 cells are then screened for various other surface markers (CD25, Foxp3, and CD127^{low}) which are characteristic of T reg cells, for example, by antibody staining, as described above and in Example 1B below.

The selected T reg cells are then cultivated and expanded, ex vivo. For example, cultivation can be done by growing cells in a growth medium adapted for T cells (e.g., RMP1-1640) after the cells have been modified and stimulated to proliferate (e.g., by exposure to allogenic, antigen producing cells treated with mitomycin C, or TGF-B1 and IL-2).

To determine if the ex vivo-modified and expanded T reg cells have comparable suppressor activity relative to the native cells (circulating in blood), these cells are tested for their ability to suppress the proliferation of certain target cells involved in the autoimmune process. This can be done using any assay involving contacting certain selected target cells (e.g., those in a mixed lymphocyte sample) with the expanded T reg cells, and measuring the target cell's ability to proliferate.

### 3. Pharmaceutical Formulation

To prepare the pharmaceutical composition, the ex vivo-modified and expanded T reg cells having suppressor activity are suspended in a pharmaceutically acceptable carrier. This can be accomplished, e.g. by washing them twice in PBS, centrifuging them, and suspending the cell pellet in the carrier.

The phrase “pharmaceutically acceptable carrier” is employed herein to refer to liquid solutions which are, within the scope of sound medical judgment, suitable for use in contact with the live T reg cells without affecting their activity, and without being toxic to the tissues of human beings and animals or causing irritation, allergic response, or other complications, commensurate with a reasonable benefit/risk ratio. A useful pharmaceutically acceptable carrier may be an injectable solution which is biocompatible with the T reg cells and does not reduce their activity or cause their death.

Non-limiting examples of materials which can serve as pharmaceutically acceptable carriers include a solvent or dispersion medium containing, for example, sterile intravenous glucose/dextrose sugar solutions, Ringer's lactate or compound sodium lactate solution.

Prevention of the action of microorganisms can be achieved by various antibacterial and antifungal agents, for example Clindamycin, Fluconazole, and/or Amphotericin B. Sterile injectable solutions of expanded T reg cells can be prepared by incorporating the live cells in the required amount in an appropriate carrier.

The number of T reg cells which can be combined with a carrier material to produce a single-dosage form will vary depending upon the subject being treated, the particular mode of administration, the degree of psoriasis symptoms, among others. Ultimately, the number of T reg cells in the pharmaceutical composition is that number that causes a therapeutic effect when administered to the patient. For example, the effective amount of the T reg cells may be about 1×10⁶ to about 1.1×10⁷ T reg cells/kg body weight, about 2×10⁶ to about 8×10⁶ T reg cells/kg body weight, about 4×10⁶ to about 7×10⁶ T reg cells/kg body weight, or about 5×10⁶ to about 7×10⁶ T reg cells/kg body weight.

### 4. Therapeutic Administration

Administration of the formulation containing the autologous T reg cells is useful to prevent or treat and/or to inhibit the activity of autoimmune, autologous, cytotoxic T and B cells in a patient suffering from psoriasis. This step comprises administering a therapeutically effective amount of autologous, modified and expanded T reg cells to the patient.

Methods of administration of the T reg cells in the pharmaceutical composition according to the disclosure described herein can be by any of a number of methods well known in the art. These methods include systemic or local administration by injection. Exemplary routes of administration include intravenous, intramuscular, intraperitoneal, or subcutaneous injection, and any combinations thereof.

The initial administering step may be a single administration or may comprise multiple administrations every, week, or 2 to 4 weeks after the initial administration. The initial administrating steps may be performed every 1 to 8 weeks. The number of initial administering steps at the start of treatment depends on the initial level of T reg cells in a patient's blood. The goal is to increase T reg cell number in the patient's peripheral blood until it is at the level of a healthy donor. This is determined by measuring the number of T reg cells in the peripheral blood of the patient before and after administering the autologous, modified and expanded T reg cells, and comparing that number to the number of T reg cells in the peripheral blood of a healthy patient. At the start of therapy, 1 to 8, 2 to 7, 3 to 6, 4 to 6, or 3 to 5 T reg cell injections can be administered every 2 to 4 or 3 to 4 weeks.

In addition, in some cases, an additional administering step may be performed every 3 to 6 months after the start of treatment, or at the end of the administration of the initial multiple treatments. However, a physician may determine that administration of the autologous T reg cells may be daily, weekly, or monthly. Even a single bolus provided during the remission stage can significantly improve the patient's condition.

In order to determine the number of T reg cells in a patient's blood, a sample is taken for measurement. Any method that enables the measurement of the number of T reg cells can be used. For example, the flow cytometry analysis can be performed. Measurement of the number of T reg cells can be done before and after each initial and secondary administering step(s), and further, can be done months after the initial; and any secondary administering step(s), for example, every two months. The modified and expanded T reg cell-containing pharmaceutical composition can also be administered as part of a combination therapy with other agents to prevent or treat psoriasis.

“Combination therapy” refers to any form of administration combining two or more different therapeutic compounds useful for treating psoriasis, where the second compound is administered while the previously administered T reg cells are still effective in the body (e.g., the two therapeutics are simultaneously effective in the patient, which may include synergistic effects of the two compounds). For example, the different therapeutic compounds can be administered in separate formulations, either simultaneously or sequentially. Thus, a patient who receives such treatment can have a combined (conjoint) effect of different therapeutic compounds.

The following examples provide specific exemplary methods of the invention, and are not to be construed as limiting the invention to their content.

### EXAMPLES

### Example 1

### Isolation and Ex Vivo-Modification and Expansion of T Reg Cells

All the manipulations are performed under aseptic conditions in a Laminar Flow Class II Biosafety Cabinet which is located in a sterile clean room following to GMP regulations.

### A. Blood Drawing

Peripheral blood (40 ml-50 ml) was taken from the ulnar vein of patients and placed into sterile (Vacutainer, BD, USA). 20 ml-30 ml blood (vacutainer glass serum tubes) was kept at room temperature (RT) for 2 hours, and then centrifuged at 350 g for 15 min. The supernatant was collected into sterile tubes (Falcon, 15 ml conical tubes), which were incubated for 40 min at 56° C. to inactivate complement. The serum was bottled in 1.5 ml vials (Coming, USA) and frozen at −20° C.

### B. Isolation of MNC's

The blood was transferred from tubes with the anticoagulant into 50 ml tubes, dilute 1:1 with Phosphate-buffered Saline (PBS, Ca⁺² Mg⁺² free, Gibco, United Kingdom). In order to separate lymphocytes, 35 ml MNC suspension was layered over 15 ml of a gradient solution (LimphoSep, d=1.077 g/ml, MP Biomedicals, USA) in 50 ml conical tubes (Falcon, USA). The tubes were centrifuged at 400 g for 30 min at 20° C. The upper layer was aspirated off, leaving the MNC layer, which was transferred to new 50 ml conical tubes. The tubes were filled with buffer and centrifuged at 300 g for 10 min. The cell pellet was resuspended in 50 ml PBS, combined in one tube, and then centrifuged at 300 g, 20° C. for 10 min. This procedure was repeated, and the cell pellet was resuspended in an appropriate amount of buffer.

For an estimation of initial CD4⁺CD25⁺Foxp3⁺CD27^{low} T reg cell numbers, the MNC population was stained with anti-CD4⁺, anti-CD25⁺, anti-Foxp3⁺, and anti-CD127⁺ mAbs (Miltenyi Biotec, Germany; eBioscience, USA). The cells were detected by flow cytometry using a MACsQuant (Miltenyi Biotec, Germany).

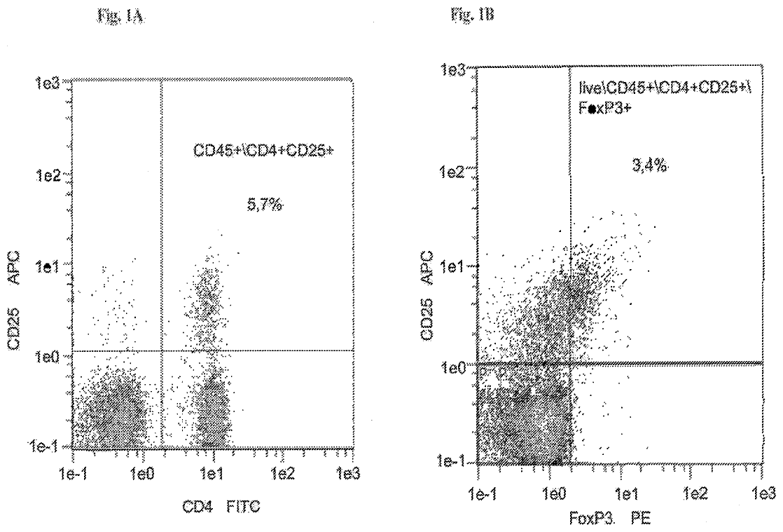

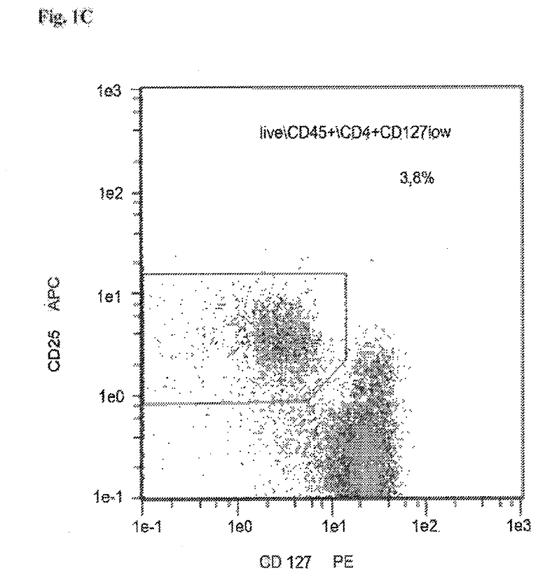

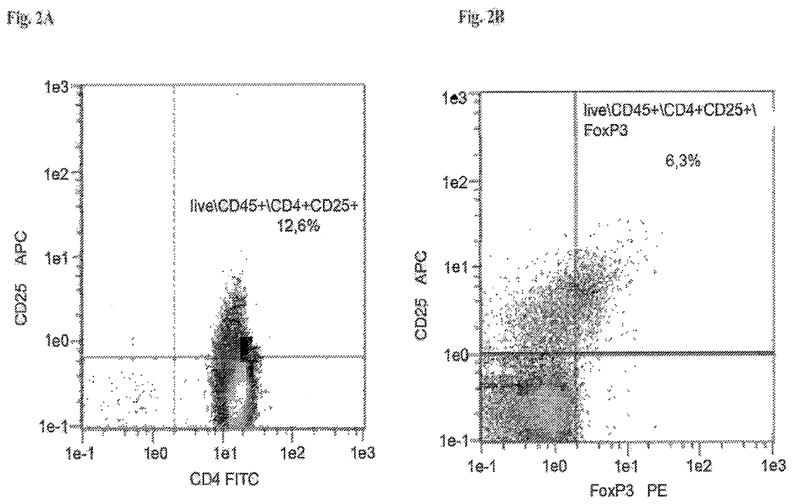

FIGS. 1A-1C show a representative example of characteristic T reg cell markers in the donor's MNCs: 5.7% of CD4⁺ T cells co-expressed CD25⁺ (FIG. 1A); 3.4% of CD4⁺ T cells co expressed Foxp3 (FIG. 1B); and 3.8% of CD4⁺ T cells co-expressed CD127^{low} (FIG. 1C).

### C. Isolation of CD4⁺ T Cells

In order to isolate CD4⁺ T cells, MNC were magnetically labeled with CD4⁺ mAbs according to the MACS Miltenyi Biotec (Germany) procedure. The immune phenotype of isolated CD4⁺ T cells was estimated by flow cytometry. In average, 94±4% (n=19) of the isolated cells were CD4⁺ T cells.

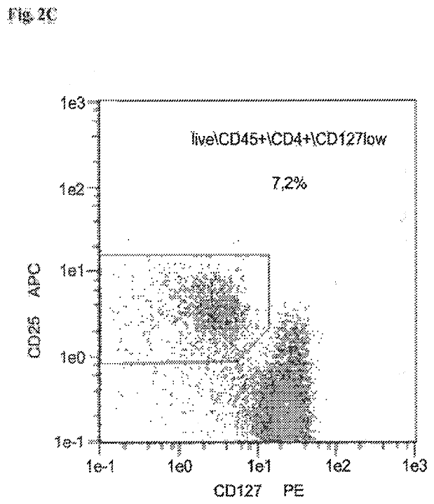

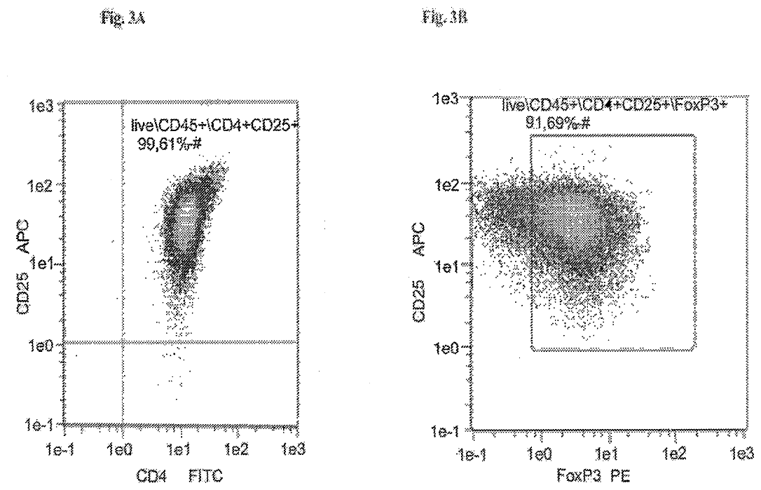

Expression of T cell markers on these cells is shown in FIGS. 2A-2C from one representative experiment (total 19): 97.5% of cells expressed CD4+, and 12.6% of these CD⁴⁺ cells co-expressed CD25⁺ (FIG. 2A); 6.3%% of these CD4+ cells co-expressed Foxp3⁺ (FIG. 2B); and 7.2% of these CD4+ cells co-expressed CD127^{low} (FIG. 2C).

### D. Modification and Expansion of T Reg Cells

The medium used for the T reg cell culture was RPMI-1640 which contains phenol red, L-glutamine, and 25 MM HEPES (Gibco, UK) with the addition of both 5-10% autologous serum and 1% pen/strep (Gibco, UK). This medium was supplemented with 1 ng/ml-50 ng/ml transforming growth factor 1 (TGF 1) (R&D Systepsoriasis, UK), 10 U/ml-1000 U/ml interleukin-2, (IL-2, R&D Systepsoriasis, UK), 0.1 μg/ml-10 μg/ml mouse anti-human CD3 mAbs (Med biospecter, RF), and 0.1 μg/ml-10 μg/ml mouse anti-human CD28 mAbs (BD Pharmingen, USA). Expanded CD4⁺ T cells were cultured at 37° C. in 5% CO₂ for 6 to 8 days in flasks (either 25 cm² or 75 cm²) with all supplements. After 3 to 4 days, IL-2, TGFB1, anti-human CD3mAbs, and anti-human CD28 mAbs were added.

D. Phenotypic Characterizations of T Reg Cells after Modification and Expansion Ex Vitro

Modified and expanded cells were characterized at the end of culture. Flow cytometry was used to estimate the total numbers of live cells and the proportion of CD4⁺CD25⁺Foxp3⁺CD27^{low} cells in the cell suspension. To assure that the endotoxin levels in cell preparations were negligible, aliquots were tested with the Limulus assay kit (Sigma-Aldrich, USA), according to the manufacturer's protocols.

TABLE 1

| | | Marker |
|---|---|---|
| | Marker | Measurement |
| | Measurement | in Modified and |
| Markers | in Initial cells | Expanded cells |
| CD4⁺ | 93.9 ± 4.5 | 99.8 ± 0.2 |
| CD4⁺CD25^{hi} | 15.7 ± 4.0 | 95.9 ± 2.4 |
| CD4⁺CD25⁺CD62L⁺ | 18.8 ± 9.0 | 54.6 ± 3.8 |
| CD4⁺CD25⁺Foxp3⁺ | 6.1 ± 4.8 | 89.6 ± 3.2 |
| CD4⁺CD25⁺CD152⁺ | 5.4 ± 2.7 | 93.8 ± 3.0 |
| CD4⁺CD25⁺CD127^{low} | 6.7 ± 4.1 | 91.3 ± 3.2 |

Table 1 shows the results of flow cytometry of initial CD4⁺ T cells and the same cells after 6 to 7 days of culture with treating and stimulating molecules.

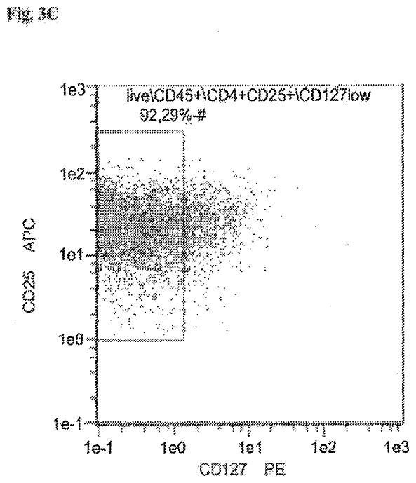

FIGS. 3A-3C show a representative sample of T reg cells expression after 6 days of ex vitro culture. 99.6% CD4⁺ T cells co-expressed CD25hi (FIG. 3A); 91.7% CD4⁺ T cells co-expressed Foxp3 (FIG. 3B); and 92.3% CD4⁺ cells co-expressed CD127^{low} (FIG. 3C).

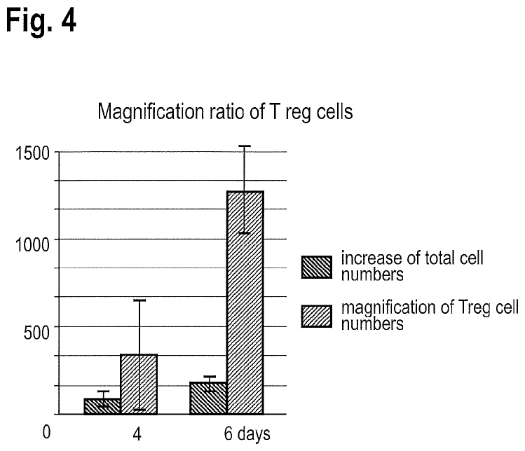

During the 6 days of propagating CD4 T cells (obtained from 19 donors), the total amount of cells increased 27.2±7.3×, whereas the number of T reg cells CD4⁺CD25⁺Foxp3⁺ increased 1272±470× (FIG. 4).

### E. Functional Capacity of Modified and Expanded T Reg Cells

To determine if autologous ex vivo-modified and expanded T reg cells keep their own suppressive ability, their suppressive capacity to inhibit proliferation of target cells in a mixed lymphocyte reaction (MLR) was compared initially and after the expansion of T reg cells.

To this end, autologous target cells (CD4⁺, CD4⁺CD25) are isolated using the magnetic beads selection method (Miltenyi Biotec), stained with carboxyfluorescein succinimidyl ester (CFSE, Fluka, USA), and cultivated with or without equal numbers (1:1) of native T reg cells isolated from human blood or induced, ex vivo-modified and expanded T reg cells. Either T cells CD4⁺CD25⁻ T cells or CD4⁺ T cells were stimulated by 5 μg/ml (CD3 mAbs and allogeneic MNC treated with mitomycin C and depleted of CD3⁺ T cells by the magnetic bead selection method (Miltenyi Biotec).

After 4 to 5 days of culture, cell proliferation is estimated by measurement of a reduction of 5(6) Carboxyfluorescein diacetate N-succinimidyl ester (CFSE) in proliferating cells.

The functional activity of T reg cells isolated from the peripheral blood of psoriasis patients is found to be substantially reduced.

### Example 2

### Treatment of Psoriasis Patients with Ex Vivo Modified and Expanded T Reg Cells

Five patients with psoriasis in the remission stage are treated with autologous, ex vivo-modified and expanded CD4⁺CD25⁺CD127^{low}T reg cells.

Patients undergoing treatment do not receive either steroids or immunotherapy for at least 3 consecutive months. All the patients signed Consent Agreement before taking part in the study.

Increased numbers of circulating T reg cells are shown in patients 4 days after injection of autologous, ex vivo-modified and expanded T reg cells. In addition, a reduction or psoriatic symptoms is seen.

These results obtained demonstrate that treatment with autologous, ex vivo-modified and expanded T reg cells during the remission phase improved efficacy and reduced side effects of conventional treatment in patients with psoriasis.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating and preventing psoriasis in a mammalian subject, comprising administration to the subject a pharmaceutical formulation comprising:
a therapeutically effective amount of ex vivo-modified and expanded, autologous regulatory CD4⁺CD25⁺Foxp⁺CD127^{low} T cells (T reg); and
a pharmaceutically acceptable carrier.

2. The method of claim 1, wherein T reg cells have been derived from the peripheral blood of the subject to be treated.

3. The method of claim 1, wherein T reg cells have been treated with a CD3 mAb, a CD28 mAb, TGF-1, and IL-2.

4. The method of claim 1, wherein the T reg cells are present at 1×10⁶ to 1.1×10⁷ T reg cell/kg body weight of the subject to be treated.

5. The method of claim 4, wherein the T reg cells are present at 2×10⁶ to 8×10⁶ T reg cells/kg body weight of the subject to be treated.

6. The method of claim 4, wherein the T reg cells are present at 4×10⁶ to 7×10⁶ T reg cells/kg body weight of the subject to be treated.

7. The method of claim 4, wherein the T reg cells are present at 5×10⁶ to 7×10⁶ T reg cells/kg body weight of the subject to be treated.

8. The method of claim 1, wherein the carrier is an intravenous glucose/dextrose solution, Ringer's lactate solution, sodium lactate solution, or Rheopolyglukin.

9. The method of claim 1, further comprising administering another psoriasis therapeutic compound.
